(19) United States
(12) Patent Application Publication
(10) Pub. No.: US 2026/0001823 A1
(43) Pub. Date: Jan. 1, 2026

(54) PROCESS FOR CONVERTING OLEFINS TO JET FUEL WITH STRIPPED DILUENT OIL

(71) Applicant: UOP LLC, Rosemont, IL (US)

(72) Inventors: Debanjan Chakrabarti, Arlington Heights, IL (US); Manuela Serban, Northbrook, IL (US); Kyle Krynski, Chicago, IL (US); Ian G. Horn, Streamwood, IL (US); Jeannie Blommel, Oregon, WI (US)

(21) Appl. No.: 19/246,957

(22) Filed: Jun. 24, 2025

Related U.S. Application Data
(60) Provisional application No. 63/666,194, filed on Jun. 30, 2024.

(51) Int. Cl. C07C 2/74; C10L 1/04

(52) U.S. Cl. CPC C07C 2/74; C10L 1/04

(57) ABSTRACT

A process for oligomerizing olefins to distillate fuels in the presence of a diluent for absorbing the exotherm. Oligomerization may comprise oligomerizing a charge olefin stream in a first-stage oligomerization reactor which is oligomerized in a second-stage oligomerization reactor to provide an oligomerate stream. The oligomers are hydrogenated and stripped to provide a stripped distillate stream. The diluent may be taken from the stripped distillate stream.

### FIELD

The field is the conversion of olefins to distillate. The field may particularly relate to oligomerizing olefins to distillate fuels.

### BACKGROUND

Molecular sieves such as microporous crystalline zeolite and non-zeolitic catalysts, particularly silicoaluminophosphates (SAPO), are known to promote the conversion of oxygenates such as methanol to light olefins. The highly efficient Methanol to Olefins (MTO) process may convert oxygenates to light olefins and was typically considered for plastics production. Light olefins produced from the MTO process are highly concentrated in ethylene and propylene and also contain significant concentrations of butenes, pentenes, and hexenes. When methanol derived from low carbon intensity feedstocks such as carbon dioxide or municipal solid waste is fed to an MTO unit, renewable light olefins are produced.

Ethylene can be dimerized and oligomerized into olefins such as C4, C6 and C8 olefins. Propylene can be dimerized and oligomerized into olefins such as C6, C9 and C12 olefins. Ethylene and propylene can be co-oligomerized into olefins such as C5 and C7 olefins. Olefin oligomerization is an exothermic process that can oligomerize smaller olefins into larger olefins. More specifically, it can convert olefins including oligomerized olefins into a distillate including jet fuel and diesel range products. The oligomerized distillate can be saturated for use as transportation fuels.

Unlike automobile engines, jet engines cannot be replaced easily by electrical motor systems because a high energy density is required to fuel planes which cannot be supplied with batteries. Large incentives are currently available for renewable jet fuel in certain regions. Other regions have announced planned mandates for renewable jet fuel to be gradually implemented in the coming decades to meet carbon dioxide emission reduction targets.

Measures must be taken to manage the exotherm generated in the oligomerization reactions, particularly the ethylene oligomerization. Diesel product has been recycled to dilute ethylene oligomerization reactions to absorb the exotherm and dilute the oligomerization reactions occurring in the reactor.

An efficient process is desired for converting renewable olefinic feeds to distillate fuels and for managing the exotherm from ethylene oligomerization.

### BRIEF SUMMARY

We have formulated a process for oligomerizing an olefin stream to distillate fuel. The process comprises oligomerizing a charge olefin stream with an oligomerization catalyst to produce an oligomerized olefin stream. The oligomerized stream is hydrogenated to make a distillate product. The distillate product is stripped to drive off volatiles and a diluent stream is taken from a stripped bottoms stream and charged with the olefin stream to absorb and manage the exotherm generated in the oligomerization and hydrogenation reactors.

### BRIEF DESCRIPTION OF THE DRAWINGS

FIG. 1 is a schematic drawing of an oligomerization section of a process and apparatus of the present disclosure.

FIG. 2 is a schematic drawing of a hydrogenation section of a process and apparatus of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripping columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T90” or “T95” means the temperature at which 5 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86. The term “green diesel” means diesel comprising hydrocarbons not sourced from fossil fuels.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

### Detailed Description

The process disclosed involves oligomerizing an olefin stream comprising ethylene and/or propylene. Oligomerization particularly of ethylene generates a large exotherm that must be managed. The process takes a diluent stream from a stripped bottoms stream and charges it to the oligomerization reactors with the olefin stream. The diluent comprises jet and diesel range hydrocarbon species, which are expected to have improved viscosity and lower fouling potential than only diesel range hydrocarbon species. Using only diesel material for diluent pushed the jet reboiler temperature to 750 to 800° F. It is proposed to employ jet and diesel components for the diluent stream instead of only diesel range hydrocarbon species to improve viscosity and lower fouling potential by enabling a stripper column to reboil at 650 to 700° F. instead of reboiling the jet fractionation column.

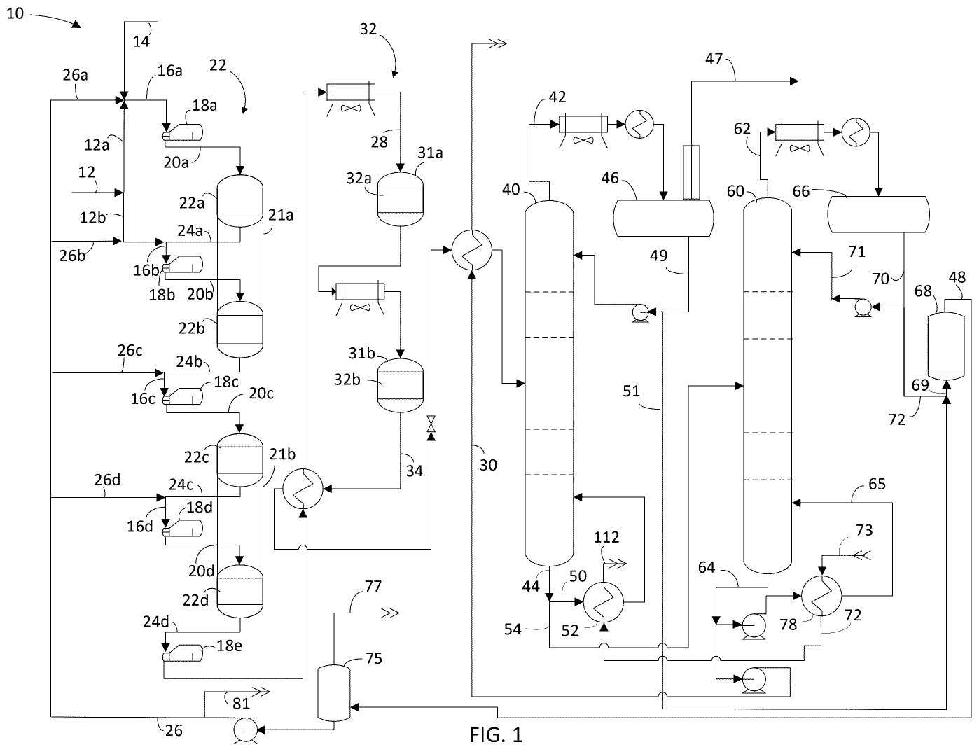

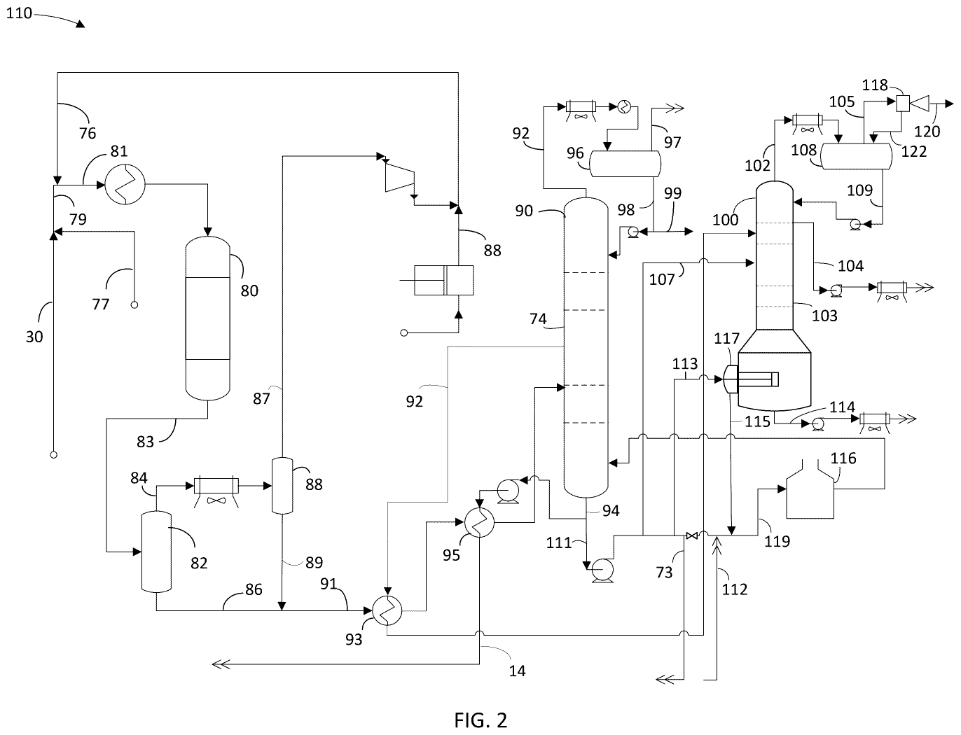

The process and apparatus may include an oligomerization section 10 illustrated in FIG. 1 and a hydrogenation section 110 as illustrated in FIG. 2.

Turning to the oligomerization section 10 of FIG. 1, a preliminary vapor olefin stream comprising C2 olefins that has been dried and compressed to provide a light olefin stream in line 12 may be charged to an oligomerization unit 10. In an aspect, a preliminary liquid olefin stream comprising C3 olefins may be also combined with the preliminary vapor olefin stream to provide the light olefin stream in line 12.

The vapor olefin stream may comprise substantial ethylene and the liquid olefin stream may comprise substantial propylene. The olefin streams may predominantly comprise ethylene and/or propylene. In an aspect, the vapor olefin stream may comprise at least 95 mol % ethylene and the liquid olefin stream may comprise at least 60 mol % and suitably at least 75 mol % propylene. The vapor olefin stream in line 12 may be styled a light olefin stream. Additional olefinic species with carbon numbers ranging from C4 to C8 can be expected in the charge streams. The light olefin streams may be provided by the dehydration of ethanol or provided from a MTO unit. The light olefin stream may be at a temperature of about 20° C. (68° F.) to about 150° C. (302° F.) and a pressure of about 2.16 MPag (350 psig), preferably about 3.5 MPag (500 psig), to about 8.4 MPag (1200 psig).

The light olefin stream may be initially contacted with a first-stage oligomerization catalyst to oligomerize the ethylene and propylene to oligomers and then contacted with a second oligomerization catalyst to oligomerize unconverted ethylene and propylene from the first-stage oligomerization.

The oligomerization reaction generates a large exotherm. For example, dimerization of ethylene can generate 612 kcal/kg (1100 BTU/lb) of heat. Consequently, this large exotherm must be managed. Accordingly, the light olefin stream in line 12 may be split into multiple olefin streams. In FIG. 1, the light olefin stream is split into two separate streams. The compressed vapor olefin stream in line 12 may be split into a first vapor olefin stream in line 12a and a second vapor olefin stream in 12b. More or less separate multiple olefin streams may be used. Up to six charge olefin streams are readily contemplated.

The vapor olefin stream in line 12 may be split into equal aliquot multiple olefin streams in lines 12a and 12b. Alternatively, the vapor olefin stream in line 12 may be split into unequal streams. In an embodiment, the vapor olefin stream may be split into two streams of equal flow rates, each comprising 50 vol % of the charge olefin stream.

To manage the exotherm, the charge olefin stream may be diluted with a diluent stream to provide a diluted olefin stream to absorb the exotherm. The diluent stream may comprise a paraffin stream in a diluent line 14. The diluent stream in the diluent line 14 may be added to the first charge olefin stream in the first charge olefin line 12a before they are charged to the first-stage oligomerization reactor 22. Preferably, the diluent stream is added to the first charge olefin stream in line 12a after the split of the charge olefin stream in line 12 into multiple olefin streams to provide a first diluted olefin charge stream in line 16a, so the diluent stream passes through all of the first-stage oligomerization reactions. Alternatively, the diluent stream may also be split into multiple streams with each diluent stream added to one or more of a corresponding charge olefin stream. The diluent stream may have a mass flow rate of about 2 to about 8 times and preferably about 3 to about 6 times the combined mass flow rates of the first charge olefin stream in the first charge olefin line 12a and the second charge olefin stream in the second charge olefin line 12b.

A recycle olefin stream in a recycle line 26 comprising C4 to C8 olefins may be mixed with the charge olefin stream and oligomerized in the first-stage oligomerization reactor 22. In an embodiment, the recycle olefin stream in line 26 is split into a plurality of recycle olefin streams 26a-26d. A recycle olefin stream in a first recycle olefin line 26a may be mixed with the first charge olefin stream in line 12a and charged to the first-stage oligomerization reactor 22. In a further embodiment, the first recycle olefin stream in the first recycle olefin line 26a is mixed with the first charge olefin stream in line 12a and the diluent stream in line 14 to provide a diluted first charge olefin stream in line 16a.

The first diluted charge olefin stream may comprise no more than 50 wt % olefins, suitably no more than 30 wt % olefins and preferably no more than 20 wt % olefins. In an embodiment, the first diluted olefin stream comprises about 10 to about 35 wt % C2 to C8 olefins. The first diluted olefin stream may comprise no more than 50 wt % ethylene, suitably no more than 25 wt % ethylene and preferably no more than 20 wt % ethylene. In an embodiment, the first diluted charge olefin stream comprises about 10 to about 20 wt % propylene. The first diluted charge olefin stream may comprise no more than 50 wt % propylene, suitably no more than 25 wt % propylene and preferably no more than 20 wt % propylene. In an embodiment, the first diluted charge olefin stream comprises about 10 to about 20 wt % propylene.

The first-stage oligomerization reactor 22 may comprise a series of first-stage oligomerization catalyst beds 22a, 22b, 22c and 22d each for charging with olefin charge streams. The first-stage oligomerization 22 reactor preferably contains four fixed first-stage oligomerization catalyst beds 22a, 22b, 22c and 22d. It is also contemplated that each first-stage oligomerization catalyst bed 22a, 22b, 22c and 22d may be in a dedicated first-stage oligomerization reactor or multiple first-stage oligomerization catalyst beds may be in two or more separate first-stage oligomerization reactor vessels. Up to six, first-stage oligomerization catalyst beds are readily contemplated. In FIG. 1, two, first stage oligomerization reactor vessels 21a and 21b are utilized.

A parallel first-stage oligomerization reactor may be used when the first-stage oligomerization reactor 22 has deactivated during which the first-stage oligomerization reactor 22 is regenerated in situ by combustion of coke from the catalyst. In another embodiment, each first-stage oligomerization reactor may comprise a lead reactor, a lag reactor and a spare reactor to facilitate regeneration. Only two reactor vessels 21a, 21b are shown in FIG. 1.

The diluted first charge olefin stream in line 16a may be cooled in a first charge cooler 18a to provide a cooled diluted first charge olefin stream in line 20a and charged to a first bed 22a of first-stage oligomerization catalyst in the first, first-stage oligomerization reactor vessel 21a of the first-stage oligomerization reactor 22. The cooled diluted first charge olefin stream in line 20a may be charged at a temperature of about 150° C. (302° F.) to about 230° C. (446° F.) and a pressure of about 3.1 MPag (450 psig) to about 8.4 MPag (1200 psig). The charge cooler 18a may comprise a steam generator.

The diluted first charge olefin stream may be charged to the first, first-stage catalyst bed 22a in line 20a preferably in a down flow operation. However, upflow operation may be suitable. The diluted first charge olefin stream is in a mixed vapor-liquid phase in which the vapor phase predominantly comprises ethylene. As oligomerization of ethylene, propylene and recycle olefins occurs in the first, first-stage oligomerization catalyst bed 22a, an exotherm is generated due to the highly exothermic nature of the olefin oligomerization reaction. Oligomerization of the first charge olefin stream produces a first oligomerized stream in a first oligomerized line 24a at an elevated outlet temperature despite the cooling and dilution. The elevated outlet temperature may be limited to between 11° C. (20° F.) and about 50° C. (90° F.) above the inlet temperature to the catalyst bed 22a.

The second charge olefin stream in line 12b may be mixed with a second recycle olefin stream in a second recycle olefin line 26b and with the first oligomerized stream in the first oligomerized line 24a removed from the first, first-stage oligomerization catalyst bed 22a in the first, first-stage reactor 21a to provide a mixed second charge olefin stream in line 16b. The first oligomerized stream in line 24a includes the diluent stream from diluent line 14 added to the first charge olefin stream in line 12a. The second charge olefin stream may comprise no more than 35 wt % C2 to C8 olefins, suitably no more than 25 wt % C2 to C8 olefins and preferably no more than 20 wt % C2 to C8 olefins. The second charge olefin stream may comprise no more than 30 wt % ethylene, suitably no more than 25 wt % ethylene and preferably no more than 20 wt % ethylene. The second charge olefin stream may comprise no more than 30 wt % propylene, suitably no more than 25 wt % propylene and preferably no more than 20 wt % propylene. The second mixed charge olefin stream in line 16b may be cooled in a second charge cooler 18b which may be located externally to the first, first-stage oligomerization reactor 21a to provide a cooled second charge olefin stream in line 20b and charged to a second bed 22b of first-stage oligomerization catalyst in the first, first-stage oligomerization reactor 21a. The charge cooler 18b may comprise a steam generator.

The second cooled charge olefin stream in line 20b may be charged at a temperature of about 150° C. (302° F.) to about 230° C. (446° F.) and a pressure of about 3.1 MPag (450 psig) to about 8.4 MPag (1200 psig). The second cooled charge olefin stream will include diluent and olefins from the first oligomerized stream. The diluted second charge olefin stream is in a mixed vapor-liquid phase in which the vapor phase predominantly comprises ethylene. The olefins from the first oligomerized stream will oligomerize in the second, first-stage catalyst bed 22b. Oligomerization of ethylene, propylene, recycle olefins and oligomers in the second olefin stream in the second, first-stage oligomerization catalyst bed 22b produces a second oligomerized olefin effluent stream in a second oligomerized line 24b at an elevated outlet temperature. The elevated outlet temperature may be limited to between 11° C. (20° F.) and about 50° C. (90° F.) above the inlet temperature to the catalyst bed 22b.

The second oligomerized stream in line 24b removed from the second, first-stage oligomerization catalyst bed 22b in the first, first-stage reactor vessel 21a may be mixed with a third recycle olefin stream in a third recycle olefin line 26c to provide a first recycle olefin charge stream in line 16c. In an embodiment, none of the first charge olefin stream in line 12a and the second charge olefin stream in line 12b is directly added to the first recycle olefin charge stream in line 16c. Alternatively, a portion of the charge olefin streams in lines 12a and 12b may be charged with the second oligomerized stream with the first recycle olefin charge stream in line 16c. The second oligomerized stream in line 24b includes the diluent stream from diluent line 14 added to the first charge olefin streams in line 12a. The first recycle olefin charge stream in line 16c may comprise no more than 30 wt % ethylene, suitably no more than 25 wt % ethylene and preferably no more than 20 wt % ethylene. The first recycle olefin charge stream may comprise no more than 30 wt % propylene, suitably no more than 25 wt % propylene and preferably no more than 20 wt % propylene. The first recycle olefin charge stream in line 16c may comprise no more than 30 wt % C2 to C8 olefins, suitably no more than 25 wt % C2 to C8 olefins and preferably no more than 20 wt % C2 to C8 olefins. The first recycle olefin charge stream in line 16c may be cooled in a third charge cooler 18c which may be located externally to the oligomerization reactor 21b to provide a cooled first recycle olefin charge stream in line 20c and charged to a third bed 22c of first-stage oligomerization catalyst in the first-stage oligomerization reactor 21b. In an embodiment, the third bed 22c of first-stage oligomerization catalyst is provided in a second, first-stage oligomerization reactor vessel 21b. The charge cooler 18c may comprise a steam generator.

The cooled first recycle olefin charge stream in line 20c may be charged at a temperature of about 150° C. (302° F.) to about 230° C. (446° F.) and a pressure of about 3.1 MPag (450 psig) to about 8.4 MPag (1200 psig). The first recycle olefin charge stream will include diluent and olefins from the second oligomerized olefin stream and the third recycle olefin stream. The olefins will oligomerize in the third catalyst bed 22c. Oligomerization of ethylene and propylene and oligomerization of oligomers in the first recycle olefin charge stream in the third bed 22c of first-stage oligomerization catalyst produces a third oligomerized stream in a third oligomerized line 24c at an elevated outlet temperature. In an embodiment, the third oligomerized stream is a penultimate oligomerized stream and the third oligomerized line 24c is a penultimate oligomerized line 24c. The elevated outlet temperature is limited to between 11° C. (20° F.) and about 50° C. (90° F.) above the inlet temperature to the catalyst bed 22c.

The third oligomerized stream in line 24c removed from the third, first-stage oligomerization catalyst bed 22c in the second first stage oligomerization reactor vessel 21b may be mixed with the fourth recycle olefin stream in line 26d to provide a second recycle olefin charge stream in line 16d. The third oligomerized stream in line 24c includes the diluent stream from diluent line 14 added to the first olefin stream in line 12a. None of the charge olefin streams in lines 12a and 12b is directly added to the second recycle olefin charge stream in line 16d. In an embodiment, the third oligomerized stream in line 24c may also be mixed with a portion of the charge olefin streams in lines 12a and 12b and be oligomerized therewith. The second recycle olefin charge stream may comprise no more than 35 wt % C2 to C8 olefins, suitably no more than 30 wt % C2 to C8 olefins and preferably no more than 25 wt % C2 to C8 olefins. The second recycle olefin charge stream may comprise no more than 30 wt % ethylene, suitably no more than 25 wt % ethylene and preferably no more than 20 wt % ethylene. The second recycle olefin charge stream may comprise no more than 30 wt % propylene, suitably no more than 25 wt % propylene and preferably no more than 20 wt % propylene. The second recycle olefin charge stream in line 16d may be cooled in a fourth charge cooler 18d which may be located externally to the second vessel 21b of the first-stage oligomerization reactor 22 to provide a cooled second recycle olefin charge stream in line 20d and charged to a fourth bed 22d of first-stage oligomerization catalyst in the second vessel 21b of the first-stage oligomerization reactor 22. The charge cooler 18d may comprise a steam generator.

The cooled second recycle olefin charge stream in line 20d may be charged at a temperature of about 150° C. (302° F.) to about 230° C. (446° F.) and a pressure of about 3.1 MPag (450 psig) to about 8.4 MPa (g) (1200 psig). The cooled second recycle olefin charge stream in line 20d will include diluent and olefins from the third or penultimate oligomerized stream and C4-C8 olefins from the fourth recycle olefin stream. The olefins will oligomerize over the fourth catalyst bed 22d. Oligomerization of ethylene and propylene in the second recycle olefin charge stream in the fourth bed 22d of first-stage oligomerization catalyst produces a fourth oligomerized stream in a fourth oligomerized line 24d at an elevated outlet temperature. The elevated outlet temperature is limited to between 11° C. (20° F.) and about 50° C. (90° F.) above the inlet temperature to the catalyst bed 22d.

The fourth oligomerized stream in line 24d exits the second reactor vessel 21b of the first-stage oligomerization reactor 22. In an embodiment, the fourth oligomerized stream in line 24d is a last oligomerized stream, and the fourth oligomerized line 24d is a last oligomerized line 24d.

The first-stage oligomerization reaction takes place predominantly in the liquid phase or in a mixed liquid and gas phase at a WHSV of 0.5 to 10 hr⁻¹ on an olefin basis. We have found that across the first-stage oligomerization catalyst beds, typically 10-50 wt % ethylene in the olefin stream converts to higher olefins. The ethylene will initially dimerize over the catalyst to butenes. A predominance of the propylene and butenes in the olefins stream charged to a first-stage oligomerization catalyst bed is oligomerized. In an embodiment, at least 99 mol % of propylene and butenes in the olefins stream are oligomerized.

The first-stage oligomerization catalyst may include a zeolitic catalyst. The first-stage oligomerization catalyst may be considered a solid acid catalyst. The zeolite may comprise between about 5 and about 95 wt % of the catalyst, for example between about 5 and about 85 wt %. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. Three-letter codes indicating a zeotype are as defined by the Structure Commission of the International Zeolite Association and are maintained at http://www.iza-structure.org/databases. UZM-8 is as described in U.S. Pat. No. 6,756,030. In a preferred aspect, the first-stage oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the first-stage oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the first-stage oligomerization catalyst comprises an MTT zeolite.

The first-stage oligomerization catalyst may be formed by combining the zeolite with a binder and then forming the catalyst into pellets. The pellets may optionally be treated with a phosphorus reagent to create a zeolite having a phosphorous component between 0.5 and 15 wt-% of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark VERSAL. A preferred alumina is available from Sasol North America Alumina Product Group under the trademark CATAPAL. This material is an extremely high purity alpha-alumina monohydrate (pseudo-boehmite) which after calcination at a high temperature has been shown to yield a high purity gamma-alumina.

A suitable first-stage oligomerization catalyst is prepared by mixing proportionate volumes of zeolite and alumina to achieve the desired zeolite-to-alumina ratio. In an embodiment, the MTT content may about 5 to about 85, for example about 20 to about 82 wt % MTT zeolite, and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

In one exemplary embodiment, an MTT-type zeolite catalyst disposed on a high purity pseudo boehmite alumina substrate in a ratio of about 90/10 to about 20/80 and preferably between about 20/80 and about 50/50 is provided in a catalyst bed or more in the first-stage oligomerization reactor 22.

The first-stage oligomerization catalyst can be regenerated upon deactivation. Suitable regeneration conditions include subjecting the first-stage oligomerization catalyst, for example, in situ, to hot air at about 400 to about 500° C. To facilitate regeneration without downtime, a swing bed arrangement may be employed with an alternative first-stage oligomerization reactor. A regeneration gas stream may be admitted to the first-stage oligomerization reactor 22 requiring regeneration. The regeneration gas may comprise air with an increased or decreased concentration of oxygen. Activity and selectivity of the regenerated catalyst is comparable to fresh catalyst.

The zeolite catalyst is advantageous as a first-stage oligomerization catalyst. The zeolitic catalyst has relatively low sensitivity towards oxygenates contamination. Consequently, a smaller degree of removal of oxygenates is required of olefinic feed in line 12 if produced from an alcohol dehydration process.

The last first-stage oligomerized stream in the last first-stage oligomerized line 24d has an increased concentration of ethylene and propylene oligomers compared to the light olefin stream in line 12. The last first-stage oligomerized stream in the last first-stage oligomerized line 24d is cooled by steam generation in a steam generator 18e or by other heat exchange and further cooled by heat exchange against a second stage oligomerized stream in line 34 and perhaps further cooled to provide a charge first-stage oligomerized stream and charged to a second-stage oligomerization reactor 32 in a second-stage oligomerization charge line 28. To achieve the most desirable olefin product, the second-stage oligomerization reactor 32 is operated at a temperature from about 80° C. (176° F.) to about 200° C. (392° F.). The second-stage oligomerization reactor 32 is run at a pressure from about 2.1 MPa (300 psig) to about 7.6 MPa (1100 psig), and more preferably from about 3.5 MPa (500 psig) to about 6.9 MPa (1000 psig). The second-stage oligomerization charge stream oligomerizes in a mixed vapor-liquid phase to predominantly C4+ olefins.

The second-stage oligomerization reactor 32 may be in downstream communication with the first-stage oligomerization reactor 22. The second-stage oligomerization reactor 32 preferably operates in a down flow operation. However, upflow operation may be suitable. The second-stage oligomerization charge stream is contacted with the second-stage oligomerization catalyst causing the unconverted ethylene from the first-stage oligomerization reactor 22 to dimerize and trimerize while higher olefins also dimerize, trimerize and tetramerize to provide distillate range olefins. With regard to the second-stage oligomerization reactor 32, process conditions may be selected to produce a higher percentage of jet range olefins which, when hydrogenated in a subsequent step as will be described below, result in a desirable jet-range hydrocarbon product. The predominance of the unconverted ethylene from the first-stage oligomerization reactor 22 is dimerized, trimerized and tetramerized. In an embodiment, at least 99 wt % of ethylene in the second-stage oligomerization charge stream is converted to mostly butenes.

The second-stage oligomerization reactor 32 may comprise a first reactor vessel 31a comprising a first bed 32a of second-stage oligomerization catalyst and a second reactor vessel 31b comprising a second bed 32b of second-stage oligomerization catalyst. A first, second-stage oligomerized stream is discharged from the first, second-stage reactor vessel 31a, cooled and charged to the second, second-stage reactor vessel 31b. A second-stage oligomerized stream with an increased average carbon number greater than the charge second-stage oligomerized stream in line 28 exits the second-stage oligomerization reactor 32 in line 34.

The first-stage oligomerization reactor 22 and the second-stage oligomerization reactor 32 may utilize vapor-liquid distribution trays to mix and disperse the ethylene vapor with liquid olefin and liquid paraffin to promote heat transfer and manage the exotherm.

The second-stage oligomerization catalyst is preferably an amorphous silica-alumina base with a metal from either Group VIII and/or Group VIB in the periodic table using Chemical Abstracts Service notations. In an aspect, the catalyst has a Group VIII metal promoted with a Group VIB metal. Typically, the silica and alumina will only be in the base, so the silica-to-alumina ratio will be the same for the catalyst as for the base. The metals can either be impregnated onto or ion exchanged with the silica-alumina base. Co-mulling is also contemplated. Catalysts for the present invention may have a Low Temperature Acidity Ratio of at least about 0.15, suitably of about 0.2, and preferably greater than about 0.25, as determined by Ammonia Temperature Programmed Desorption (Ammonia TPD) as described hereinafter. Additionally, a suitable catalyst will have a surface area of between about 50 and about 400 m²/g as determined by nitrogen BET.

The preferred second-stage oligomerization catalyst comprises an amorphous silica-alumina support. One of the components of the catalyst support utilized in the present invention is alumina. The alumina may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A particularly preferred alumina is available from Sasol North America Alumina Product Group under the trademark CATAPAL. This material is an extremely high purity alpha-alumina monohydrate (pseudo-boehmite) which after calcination at a high temperature has been shown to yield a high purity gamma-alumina. Another component of the catalyst support is an amorphous silica-alumina. A suitable silica-alumina with a silica-to-alumina ratio of 2.6 is available from CCIC, a subsidiary of JGC, Japan.

Another component utilized in the preparation of the second-stage oligomerization catalyst utilized in the present invention is a surfactant. The surfactant is preferably admixed with the hereinabove described alumina and the silica-alumina powders. The resulting admixture of surfactant, alumina and silica-alumina is then formed, dried and calcined as hereinafter described. The calcination effectively removes by combustion the organic components of the surfactant but only after the surfactant has dutifully performed its function in accordance with the present invention. Any suitable surfactant may be utilized in accordance with the present invention. A preferred surfactant is a surfactant selected from a series of commercial surfactants sold under the trademark “Antarox” by Solvay S. A. The “Antarox” surfactants are generally characterized as modified linear aliphatic polyethers and are low-foaming biodegradable detergents and wetting agents.

A suitable silica-alumina mixture is prepared by mixing proportionate volumes of silica-alumina and alumina to achieve the desired silica-to-alumina ratio. In an embodiment, about 75 to about 99 wt-% amorphous silica-alumina with a silica-to-alumina ratio of 2.6 and about 10 to about 20 wt-% alumina powder will provide a suitable support. In an embodiment, other ratios of amorphous silica-alumina to alumina may be suitable.

Any convenient method may be used to incorporate a surfactant with the silica-alumina and alumina mixture. The surfactant is preferably admixed during the admixture and formation of the alumina and silica-alumina. A preferred method is to admix an aqueous solution of the surfactant with the blend of alumina and silica-alumina before the final formation of the support. It is preferred that the surfactant is present in the paste or dough in an amount from about 0.01 to about 10 wt-% based on the weight of the alumina and silica-alumina.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried.

The paste or dough may be prepared in the form of shaped particulates, with the preferred method being to extrude the dough mixture of alumina, silica-alumina, surfactant and water through a die having openings therein of desired size and shape, after which the extruded matter is broken into extrudates of desired length and dried. A further step of calcination may be employed to give added strength to the extrudate. Generally, calcination is conducted in a stream of dry air at a temperature from about 260° C. (500° F.) to about 815° C. (1500° F.).

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

Typical characteristics of the amorphous silica-alumina supports utilized herein are a total pore volume, average pore diameter and surface area large enough to provide substantial space and area to deposit the active metal components. The total pore volume of the support, as measured by conventional mercury porosimeter methods, is usually about 0.2 to about 2.0 cc/gram, preferably about 0.25 to about 1.0 cc/gram and most preferably about 0.3 to about 0.9 cc/gram. Ordinarily, the amount of pore volume of the support in pores of diameter greater than 100 angstroms is less than about 0.1 cc/gram, preferably less than 0.08 cc/gram, and most preferably less than about 0.05 cc/gram. Surface area, as measured by the B.E.T. method, is typically above 50 m²/gram, e.g., above about 200 m²/gram, preferably at least 250 m²/gram, and most preferably about 300 m²/gram to about 400 m²/gram.

To prepare the second-stage oligomerization catalyst, the support material is compounded, as by a single impregnation or multiple impregnations of a calcined amorphous refractory oxide support particles, with one or more precursors of at least one metal component from Group VIII or VIB of the periodic table. The Group VIII metal, preferably nickel, should be present in a concentration of about 0.5 to about 15 wt-% and the Group VIB metal, preferably tungsten, should be present in a concentration of about 0 to about 12 wt-%. The impregnation may be accomplished by any method known in the art, such as, for example, by spray impregnation wherein a solution containing the metal precursors in dissolved form is sprayed onto the support particles. Another method is the multi-dip procedure wherein the support material is repeatedly contacted with the impregnating solution with or without intermittent drying. Yet other methods involve soaking the support in a large volume of the impregnation solution or circulating the support therein, and yet one more method is the pore volume or pore saturation technique wherein support particles are introduced into an impregnation solution of volume just sufficient to fill the pores of the support. On occasion, the pore saturation technique may be modified, so as to utilize an impregnation solution having a volume between about 10 percent less and about 10 percent more than that which will just fill the pores.

If the active metal precursors are incorporated by impregnation, a subsequent or second calcination at elevated temperatures, as for example, between 399° C. (750° F.) and 760° C. (1400° F.), converts the metals to their respective oxide forms. In some cases, calcinations may follow each impregnation of individual active metals. A subsequent calcination yields a catalyst containing the active metals in their respective oxide forms.

A preferred second-stage oligomerization catalyst of the present invention has an amorphous silica-alumina base impregnated with about 0.5 to about 15 wt-% nickel in the form of 3.175 mm (0.125 inch) extrudates and a density of about 0.45 to about 0.65 g/ml. It is also contemplated that metals can be incorporated onto the support by other methods such as ion-exchange and co-mulling.

The second-stage oligomerization catalyst can be regenerated upon deactivation. Suitable regeneration conditions include subjecting the catalyst, for example, in situ, to hot air at about 400 to about 500° C. To facilitate regeneration without downtime, a swing bed arrangement may be employed with an alternative second-stage oligomerization reactor. The regeneration gas may comprise air with an increased or decreased concentration of oxygen. Activity and selectivity of the regenerated catalyst is comparable to fresh catalyst.

Second-stage oligomerization reactions are also exothermic in nature. The last oligomerized olefin stream in line 24d includes the diluent stream from diluent line 14 added to the first charge olefin stream in the first charge olefin line 12a and carried through the first-stage oligomerization catalyst beds 22a-22d. The diluent stream is then transported into the second-stage oligomerization reactor 32 in line 28 to absorb the exotherm in the second-stage oligomerization reactor. A dedicated diluent line to the second-stage oligomerization reactor 32 is also contemplated for prompt control of exotherm rise or to cool down the second-stage oligomerization reactor 32.

When the oligomerization reaction is performed according to the above-noted process conditions, a C4 olefin conversion of greater than or equal to about 95% is achieved, or greater than or equal to 97%. The resulting second-stage oligomerized stream in line 34 includes a plurality of olefin products that are distillate range hydrocarbons.

An oligomerized olefin stream in line 34 with an increased C8+ olefin concentration compared to the charge second-stage oligomerization stream in line 28 is heat exchanged with the first-stage oligomerized stream in line 24d, let down in pressure, subsequently heat exchanged with an olefin splitter bottoms stream in line 30 and fed to a dealkanizer column 40. The oligomerized olefin stream in line 34 is at a temperature from about 160° C. (320° F.) to about 225° C. (437° F.) and a pressure of about 3.9 MPa (gauge) (550 psig) to about 7 MPa (gauge) (1000 psig).

We have found that light alkanes such as ethane and/or propane are generated in the first-stage oligomerization reactor 22 and/or the second-stage oligomerization reactor 32 which must be removed from the second-stage oligomerized stream for fuels production particularly to facilitate light olefin recycle to the first-stage oligomerization reactor 22. Light alkanes are inert and would accumulate in the recycle loop. Hence, the second-stage oligomerized stream in line 34 is dealkanized by fractionation in a dealkanizer column 40 to provide a light alkane stream and a dealkanized stream. In an embodiment, the light alkane stream is an ethane stream in which case the dealkanizer column 40 is a deethanizer column. In another embodiment, the light alkane stream is a propane stream in which case the dealkanizer column 40 is a depropanizer column. The light alkane stream may contain ethane and/or propane and can also be a mixture of ethane and propane.

In the dealkanizer column 40, light alkanes such as C3- and suitably C2-hydrocarbons, are separated perhaps in a light alkane overhead stream in an overhead line 42 from perhaps a dealkanized bottoms stream in a bottoms line 44 comprising C4+ and suitably C3+ hydrocarbons. The dealkanizer column 40 may be operated at a bottoms temperature of about 177° C. (350° F.) to about 302° C. (575° F.) and an overhead pressure of about 207 kPa (gauge) (30 psig) to about 965 kPa (gauge) (140 psig) if operated as a deethanizer column. The dealkanizer column 40 may be operated at a bottom temperature of about 194° C. (381° F.) to about 333° C. (630° F.) and an overhead pressure of about 207 kPa (gauge) (30 psig) to about 1.38 MPa (gauge) (200 psig) if operated as a dealkanizer column.

The light alkane overhead stream in the overhead line 42 may be cooled and separated in a dealkanizer receiver 46 to provide a dealkanized off-gas stream in an off-gas line 47 in which it may be chilled to further condense condensables in the off-gas stream to return back to the receiver 46. In some embodiments, the net off-gas stream 47 can undergo processing to recover the olefins, which could then be recycled back to the oligomerization reactors 22, 32. Uncondensed off-gas in the off-gas stream may be fed to further processing such as to be taken as fuel gas. Condensate from the dealkanizer receiver 46 may be refluxed back to the dealkanizer column 40 in a dealkanizer overhead liquid line 49. The dealkanized off-gas stream may be used as fuel for providing heating duty in the process 10. In an embodiment, some of the condensate from the dealkanizer receiver 44 in line 49 may be taken as a condensed olefin recycle stream in line 51 which is combined with a light olefin recycle stream in a recycle line 72 and fed to a drier 68 in line 69 to be dried before it is recycled to the first stage oligomerization reactor in lines 48 and 26.

The dealkanized stream perhaps in the bottoms line 44 may be split between a reboil stream in line 50 which is reboiled by heat exchange in heat exchanger 52 with a cooled hot oil stream in line 72 perhaps taken from a stripped bottom stream in the hot oil stream in line 73 and a net bottoms dealkanized stream in a net bottoms dealkanizer line 54 which is fed directly to an olefin splitter column 60 perhaps without heating. The reboiled bottom stream in line 50 may be returned boiling to the dealkanizer column 40 to provide heating requirements. A twice cooled, hot oil stream is taken in line 112 back to a stripper reboiler in FIG. 2. In another embodiment, feed to dealkanizer column 40 is not preheated by heat exchange with the olefin splitter bottoms stream in line 30, but by heat exchange with ethanol feed to an ethylene dehydration unit to make ethylene for the process that is provided in line 12.

The net dealkanized stream in the net bottoms dealkanizer line 54 is split by fractionation in an olefin splitter column 60 into a light olefin stream perhaps in an olefin splitter overhead line 62 and a heavy oligomerized stream perhaps in an olefin splitter bottoms line 64. Olefins may be recycled to the first-stage oligomerization reactor 22 from the olefin splitter overhead stream in the overhead splitter overhead line 62. The olefin splitter overhead stream may be chilled to about 19° C. (66° F.) to about 93° C. (200° F.) and fully condensed to provide an olefin split condensate stream in line 70. The light olefin condensate from a bottom of the olefin splitter receiver in line 70 may be split between a reflux stream that is refluxed back to the column in line 71 and the light olefin recycle stream in a recycle line 72 that may be dried in the drier 68 with the condensed olefin recycle stream in line 51 and recycled to the first-stage oligomerization reactor 22 or alternatively to the second-stage oligomerization reactor 32 in recycle line 48. The light oligomerized stream in the recycle line 72 may comprise about 1 to about 15 wt % or perhaps a predominance of the olefin split condensate stream in line 70. The drier 68 may comprise a bed of zeolite for adsorbing water which may be periodically regenerated with hot oil from the stripped bottoms steam taken from the stripped bottoms steam in line 94. The drier operates at about 16° C. (60° F.) to about 66° C. (150° F.) and a pressure of about 138 kPa (g) (20 psig) to 2 MPa (g) (300 psig). Regeneration with hot oil can occur at about 177° C. (350° F.) to about 332° C. (600° F.).

A dried, light olefin recycle stream in line 48 may comprise about 40 to about 80 wt % C4-C8 olefins. In an embodiment, the dried light olefin stream in line 48 may be flashed in a knock-out drum 75 to remove vapors in a light olefin vapor stream which may be taken as fuel gas in line 77. The bottoms stream in line 79 from the knock-out drum may be split into two streams, a light olefin recycle stream 26 and light olefin purge stream in line 81. The purge stream in line 81 is transported to the hydrogenation unit 110, and the liquid recycle olefin oligomer stream in line 26 may be recycled to the first-stage oligomerization reactor 22 to oligomerize the C4-C8 olefins.

The heavy oligomerized stream in the splitter bottoms line 64 may be split between a reboil stream in a splitter reboil line 65 that is reboiled by heat exchange in an olefin splitter reboiler 78 with a hot oil stream in line 73 perhaps taken from the stripped bottoms stream in line 94 from FIG. 2 and fed back reboiling to the olefin splitter column 60. A cooled hot oil stream emerges from the heat exchanger 78 in line 72 before it is heat exchanged with reboiler bottom stream in the dealkanized bottoms line 50 in the dealkanizer reboiler 52. The twice cooled hot oil stream in line 112 is returned back to the hydrogenation section 110 in FIG. 2 to be reboiled and fed back to the stripper column 90. The heavy oligomerized stream in the net splitter bottoms line 30 is cooled by heat exchange with the second-stage oligomerized stream in line 34 before it is transported to the hydrogenation section 110 in FIG. 2. No purge of the heavy oligomerized stream need be taken. The heavy oligomerized stream comprises C9+ olefins that once cooled can be transported to the hydrogenation section 110.

Turning to the hydrogenation unit 110 in FIG. 2, the heavy oligomerized stream in the net olefin splitter bottoms line 30 from FIG. 1 comprising distillate-range C9+ oligomerized olefins may be hydrogenated to saturate the olefinic bonds in a hydrogenation reactor 80 to provide a distillate fuel stream. This step is performed to ensure the product fuel meets or exceeds the thermal oxidation requirements specified in ASTM D7566-20 for Alcohol to Jet Synthesized Paraffinic Kerosene (ATJ-SPK). Additionally, hydrogenating the heavy oligomerized stream will provide the paraffin stream that may be used as the diluent stream in line 14. The heavy oligomerized stream in line 30 may be combined with the light olefin liquid stream in line 77 also from FIG. 1 to produce a combined olefin stream in line 79. The combined olefin stream in line 79 may also be combined with a hydrogen stream in line 76 to provide a combined hydrogenation charge stream in line 81 which is cooled perhaps by heat exchange with a feed ethanol stream and charged to the hydrogenation reactor 80 at 125° C. (257° F.) to about 204° C. (400° F.) and 2.8 MPa (400 psig) to about 6.9 MPa (1000 psig). An excess of hydrogen may be employed to ensure complete saturation such as about 1.5 to about 5.0 of stochiometric hydrogen.

Hydrogenation is typically performed using a conventional hydrogenation or hydrotreating catalyst, and can include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to, oxides such as silica, alumina, titania, calcium carbonate, barium sulfate, and carbons. The catalyst support can be in the form of powder, granules, pellets, or the like.

In an exemplary embodiment, hydrogenation is performed in the hydrogenation reactor 80 that includes a platinum-on-alumina catalyst, for example about 0.1 wt % to about 2 wt %, preferably about 0.5 wt % to about 0.9 wt %, platinum-on-alumina catalyst. In another embodiment, the hydrogenation catalyst comprises about 5 to about 30 wt % nickel catalyst. The hydrogenation reactor 80 converts the olefins into a paraffin product having the same carbon number distribution as the olefins, thereby forming distillate-range paraffins suitable for use as jet and diesel fuel.

The hydrogenated distillate stream discharged from the hydrogenation reactor 80 in line 83 may be separated in a hot separator 82 which provides a hydrocarbon split. In the hot separator 82, the hydrogenated distillate stream is separated into a hot hydrogenated vapor stream in an overhead line 84 and a hot distillate liquid stream in the hot separator bottoms line 86. The hydrogenated distillate liquid stream in the bottoms line 86 may be combined with a cold heavy distillate liquid stream in a cold bottoms line 89 to provide a combined separator liquid distillate stream in line 91. The combined separator liquid distillate stream in line 91 may be heated by heat exchange with a stripped stream in line 92 in a stripping heat exchanger 93. The heated combined separator bottom distillate stream in the bottoms line 91 may then be further heated by heat exchange with the diluent stream in line 14 before the diluent stream is recycled to the first-stage oligomerization reactor 22 in FIG. 1. The further heated heavy liquid distillate stream in the hot bottoms line 91 may be stripped of volatiles in a stripping column 90. The hot separator may be operated at a temperature of about 204° C. (400° F.) to about 343° C. (650° F.) and a pressure of 2.8 MPa (400 psig) to about 6.9 MPa (1000 psig).

The hot vapor distillate stream in the hot overhead line 84 may be cooled and fed to a cold separator 88. The cold separator 88 separates the cooled hot vapor hydrogenated stream in the hot overhead line 84 into a cold vapor hydrogenated stream in a cold overhead line 87 and a cold heavy liquid distillate stream in the cold bottoms line 89. The cold vapor hydrogenated stream in the cold overhead line 87 may be compressed and combined with a make-up hydrogen stream in line 88 to provide the hydrogen stream in line 76. The cold liquid distillate stream in the bottoms line 89 may be combined with the hot liquid distillate stream in the hot separator bottoms line 86 to provide the combined separator liquid distillate stream in line 91 and fed to the stripping column 90. The combined separator liquid distillate stream in line 91 may be heated by heat exchange with a stripped stream in line 92 in a flash stripping heat exchanger 93 and further heated by heat exchange with the diluent stream in line 14 in a heat exchanger 95 and fed to the stripping column 90. The cold separator may be operated at a temperature of about 32° C. (90° F.) to about 71° C. (150° F.) and a pressure of about 2.8 MPa (400 psig) to about 4.5 MPa (650 psig).

The stripping column 90 may involve stripping with a reboiler to remove naphtha and lighter materials from the combined separator liquid distillate stream in line 91. The stripping column 90 removes residual light gases from the liquid distillate streams to provide a stripping overhead stream in a stripping overhead line 92 and a stripped distillate stream in a stripping bottoms line 94. The stripping overhead stream in the stripping overhead line 92 is cooled and separated in a stripping receiver 96 to provide a stripping off-gas stream in a stripping receiver overhead line 97 and a condensate stream in line 98, a part of which is refluxed to the column. The stripping off-gas stream in line 97 may be transported to the fuel gas header. The stripping off gas stream in the receiver overhead line 97 can be used to fuel the stripper heater 116 for the stripping column 90.

A reflux stream taken from the condensate stream in line 98 may be refluxed to the stripping column 90 while a wild naphtha stream is taken in line 99 from the condensate stream. The stripping column 90 may be operated at a bottoms temperature of about 232° C. (450° F.) to about 388° C. (730° F.), preferably no more than 360° C. (680° F.), and an overhead pressure of about 207 kPa (30 psig) to about 1380 kPa (200 psig).

After undergoing stripping to remove volatiles in the stripping column 90, the stripped distillate stream in the stripping bottoms line 94 comprises C9+ materials. The stripped distillate stream in the stripping bottoms line 94 may be split into several streams. A diluent stream is taken in line 14 and heat exchanged with the combined separator liquid distillate stream in line 91 in the heat exchanger 95 and recycled to the oligomerization reactor to absorb the exotherm in FIG. 1. The diluent stream in line 14 may be recycled back to be mixed with the charge olefin streams in lines 12a and 12b in the oligomerization section 10 in FIG. 1, preferably the first charge olefin stream in line 12a, to provide the first diluted olefin charge stream in line 16a to absorb the exotherm in the oligomerization reactor 22. The stripped distillate stream in the diluent line 14 is paraffinic, so it will be inert to the oligomerization and hydrogenation reactions to which it may be subject. A stripping reboil stream in line 111 is pumped and split into a net stripped stream in line 107, a jet fractionation reboiling stream in line 113 and a hot oil stream in line 73. The net stripped stream in line 107 is fed to a jet fractionation column 100 without further heating. The jet fractionation reboiling stream in line 113 is hot enough to reboil the jet fractionation column 100 and is fed thereto for this purpose. In an embodiment, the jet fractionation reboiling stream in line 113 is fed to a stabbed-in reboiler 117 to reboil the jet fractionation column and provide a cooled jet fractionation reboiling stream in line 115. The hot oil stream in line 73 is forwarded to the oligomerization section 10 in FIG. 1 to reboil the splitter reboil stream in the splitter reboil line 65 by heat exchange in the olefin splitter reboiler 71. The cooled hot oil stream from the heat exchanger in line 72 then reboils the dealkanizer reboiler bottom stream in the dealkanized bottoms line 50 in the dealkanizer reboiler 52 before it is returned back in hot oil return line 112 to the hydrogenation section 110 in FIG. 2 to be reboiled and fed back to the stripping column 90.

The cooled hot oil return stream in line 112 and the cooled jet fractionation reboiling stream in line 115 may be rejoined with the pumped stripping reboil stream in line 111 to provide a combined reboil stream in line 119 and reboiled in a heater 116 which may be a fired heater. The reboiled stripped stream in line 119 is returned to the stripping column 90. The net stripped stream in line 107, the jet fractionation reboiling stream in line 113 and the hot oil stream in line 73 are all taken from the stripping reboil stream in line 111 before it is reboiled in the combined reboil stream in line 119 by the heater 116.

An intermediate stream comprising C8+ hydrocarbons may be taken from a side 74 of the stripping column 90 in line 92. The intermediate stream typically comprises C8-C9 hydrocarbons. This intermediate steam is taken to prevent C8-C9 paraffins from separating in the stripped bottoms stream and recycling as diluent oil to the oligomerization section 10. In the oligomerization section 10, the C8 and C9 paraffins would go into the overhead of the olefins splitter column and recycle to the oligomerization reactors 22 and 32 with no way of exiting the oligomerization unit since they are inert to oligomerization. Hence, the intermediate stream is taken in line 92 from the side of the stripping column and heated by heat exchange with the combined separator liquid distillate stream in 91 in the heat exchanger 93 and fed to the jet fractionation column 100. The net stripped stream in line 107 is fed to the jet fractionation column 100 at an inlet below an elevation of the inlet for the intermediate stream in line 92.

In the jet fractionation column 100, the stripped distillate stream and the intermediate stripped stream may be separated into a jet overhead stream in an overhead line 102, an intermediate synthetic aviation fuel stream boiling in the jet fuel range in a side line 104 from a side 103 of the jet fractionation column 100 and a green diesel stream in a bottoms line 114. The green diesel stream can be used as a diesel blendstock. The jet fractionation column 100 is preferably operated at vacuum to reduce the boiling temperature for the diesel bottoms which enables it to reboil by heat exchange with the jet fractionation reboiling stream in line 113 taken from the stripped distillate stream in line 111. This also enables the jet fractionation column 100 to operate without a separate heater. The fired heater 116 can be utilized with the stripping column 90 to provide reboiling heat to the dealkanizer column 40, the olefins splitter column 60, the stripping column 90 and the jet fractionation column 100. The jet fractionation column 100 may be operated at a bottom temperature of about 288° C. (550° F.) to about 343° C. (650° F.) and an overhead absolute pressure of about 0 kPa (0 psia) to about 175 kPa (10 psia).

The jet fractionation overhead stream in the overhead line 102 may be cooled and fully condensed and fed to a jet fractionation receiver 108. The jet fractionation overhead condensate may be refluxed back to the jet fractionation column 100 in a jet fractionator overhead liquid line 109. A vacuum pump 118 may pull a vacuum on a receiver overhead line 105 from the jet fractionation receiver 108. Non-condensables can be taken in line 120 while condensables are returned to the receiver 108 in line 122.

The green jet stream taken in the side line 104 comprises kerosene range C8-C18 hydrocarbons which may be cooled and taken as a jet fuel product stream meeting applicable SPK standards. In an alternative embodiment, the green jet stream may be taken from the condensate stream in line 109 from the jet fractionation receiver 108 instead of refluxing all of the condensate to the column. This green jet stream taken from line 109 may have to be further stripped to remove light ends. In such an embodiment, no side line 104 would be taken to recover the green jet fuel stream. The green jet fuel stream may comprise synthetic aviation fuel and meet applicable qualifying standards.

The green diesel bottoms stream in the bottoms line 114 provides a diesel blendstock stream. The jet fractionation column is reboiled by the reboiler 117 by heat exchange with the jet fractionation reboiling stream 113. No heater is required for the jet fractionation column 100, so that reboiling can be effected by heat exchange. The stripped bottoms stream in the stripped bottoms line 94 is lighter than the diesel in the jet fractionation bottoms line 114. Thus, the fired heater 116 can be run at lower temperature with less chance of fouling than if it were employed to reboil predominantly diesel boiling range materials in the jet fractionation column 100.

Starting with ethylene and/or propylene, the disclosed process can efficiently produce Sustainable Aviation Fuel that meets applicable fuel requirements while managing exothermic heat generation and posing less chance of fouling in the jet fractionation column 100. The process can also produce green gasoline and diesel byproducts that may either be used as-produced or as a blending component in fuels that meet applicable fuel requirements depending on the specific application. Carbon recovery in the process can exceed 95%. Both the jet fuel stream in the side line 104 and the diesel product stream in line 114 can be cooled and fed to their respective fuel pools.

### Example

A first stage oligomerization catalyst comprising zeolite and a second stage oligomerization catalyst comprising metal were loaded in a pilot plant reactor in a stacked bed configuration. The reactor was fed primarily with ethylene, propylene, and paraffin diluent, along with smaller amounts of C4+ olefins. The test was conducted at 6.2 MPa (gauge) (900 psig) pressure, with an inlet temperature to the first oligomerization stage catalyst varying from 160 to 250° C. and to the second oligomerization stage catalyst varying from 120 to 250° C. and WHSV in the fresh olefin charge varying from 0.25 to 2.0 hr⁻¹. Depending on the test conditions, the results exhibited over 97 wt % ethylene and propylene conversion, high jet and recyclable light olefin selectivity and light paraffin selectivity which is approximately 3-4 wt % at steady state under these conditions. The majority of the light paraffins consists of ethane and propane.

The oligomerization product generated in this pilot plant run was then hydrogenated and fractionated. Table 1 displays the physical properties of jet fuel which was generated from the hydrogenated oligomerization product.

TABLE 1

| Jet Fuel Property | Value | |
|---|---|---|
| Density @ 60 F. | 768.3 | kg/m³ |
| Flash Point | 42° | C. |
| Freeze Point | <−80° | C. |

| | |
|---|---|
| Distillation Temp (D86): T10 | 164.3 |
| Distillation Temp (D86): T50 | 197 |
| Distillation Temp (D86): T90 | 263.7 |
| Distillation Temp (D86): | 285.6 |
| Final boiling point | |

### Specific Embodiments

While the following is described in conjunction with specific embodiments, it will be understood that this description is intended to illustrate and not limit the scope of the preceding description and the appended claims.

A first embodiment of the disclosure is a process for oligomerizing an olefin stream comprising oligomerizing a charge olefin stream in the presence of a diluent stream over an oligomerization catalyst to produce an oligomerized stream; hydrogenating the oligomerized stream to provide a distillate stream; stripping the distillate stream to provide a stripped stream; and taking the diluent stream from the stripped stream. An embodiment of the disclosure is one, any or all of prior embodiments in this paragraph up through the first embodiment in this paragraph wherein the oligomerizing step further comprises oligomerizing the charge olefin stream in the presence of the diluent stream in a first-stage oligomerization reactor to provide a first-stage oligomerized stream and oligomerizing the first-stage oligomerized stream over a second-stage oligomerization catalyst to provide a second-stage oligomerized stream to provide the oligomerized stream. An embodiment of the disclosure is one, any or all of prior embodiments in this paragraph up through the first embodiment in this paragraph further comprising stripping naphtha and lighter materials from C9+ hydrocarbons in the stripping step. An embodiment of the disclosure is one, any or all of prior embodiments in this paragraph up through the first embodiment in this paragraph further comprising separating an intermediate stream comprising C8+ hydrocarbons from the distillate stream in the stripping step. An embodiment of the disclosure is one, any or all of prior embodiments in this paragraph up through the first embodiment in this paragraph further comprising fractionating the intermediate stream and a jet fractionation feed stream taken from the stripped stream in a jet fractionation column. An embodiment of the disclosure is one, any or all of prior embodiments in this paragraph up through the first embodiment in this paragraph further comprising reboiling a reboil stripper bottoms stream. An embodiment of the disclosure is one, any or all of prior embodiments in this paragraph up through the first embodiment in this paragraph further comprising taking a jet fuel cut from a side or a condensed overhead of the jet fractionator column. An embodiment of the disclosure is one, any or all of prior embodiments in this paragraph up through the first embodiment in this paragraph further comprising operating the jet fractionation column at vacuum. An embodiment of the disclosure is one, any or all of prior embodiments in this paragraph up through the first embodiment in this paragraph further comprising reboiling the jet fractionator by heat exchange with a jet reboil stream taken from the stripped stream. An embodiment of the disclosure is one, any or all of prior embodiments in this paragraph up through the first embodiment in this paragraph further comprising splitting the oligomerized stream into a light olefin stream and a heavy oligomerized stream in an olefin splitter column and taking a recycle olefin stream from the light olefin stream and charging the recycle olefin stream to the oligomerizing step. An embodiment of the disclosure is one, any or all of prior embodiments in this paragraph up through the first embodiment in this paragraph further comprising reboiling the olefin splitter column by heat exchanging a heavy oligomerized stream with a hot oil stream taken from the stripped stream. An embodiment of the disclosure is one, any or all of prior embodiments in this paragraph up through the first embodiment in this paragraph further comprising dealkanizing the oligomerized stream before splitting the oligomerized stream wherein the splitting the oligomerized stream further comprises splitting a dealkanized oligomerized stream. An embodiment of the disclosure is one, any or all of prior embodiments in this paragraph up through the first embodiment in this paragraph further comprising reboiling a dealkanized stream by heat exchange with a cooled hot oil after heat exchange with the heavy oligomerized stream. An embodiment of the disclosure is one, any or all of prior embodiments in this paragraph up through the first embodiment in this paragraph wherein the hydrogenating step comprises hydrogenating the heavy oligomerized stream.

A second embodiment of the disclosure is a process for oligomerizing an olefin stream comprising oligomerizing A charge olefin stream in the presence of a diluent stream in a first-stage oligomerization reactor to provide a first-stage oligomerized stream and oligomerizing the first-stage oligomerized stream over a second-stage oligomerization catalyst to provide a second-stage oligomerized stream to provide an oligomerized stream; hydrogenating the oligomerized stream to provide a distillate stream; stripping the distillate stream to provide a stripped stream; and taking the diluent stream from the stripped stream. An embodiment of the disclosure is one, any or all of prior embodiments in this paragraph up through the second embodiment in this paragraph further comprising stripping naphtha and lighter materials from C9+ hydrocarbons in the stripping step. An embodiment of the disclosure is one, any or all of prior embodiments in this paragraph up through the second embodiment in this paragraph further comprising separating an intermediate stream comprising C8+ hydrocarbons from the distillate stream in the stripping step.

A third embodiment of the disclosure is a process for oligomerizing an olefin stream comprising oligomerizing a charge olefin stream in the presence of a diluent stream over an oligomerization catalyst to produce an oligomerized stream; hydrogenating the oligomerized stream to provide a distillate stream; stripping the distillate stream to provide a stripped stream and an intermediate stream comprising C8+ hydrocarbons; and taking the diluent stream from the stripped stream. An embodiment of the disclosure is one, any or all of prior embodiments in this paragraph up through the third embodiment in this paragraph wherein the oligomerizing step further comprises oligomerizing the charge olefin stream in the presence of the diluent stream in a first-stage oligomerization reactor to provide a first-stage oligomerized stream and oligomerizing the first-stage oligomerized stream over a second-stage oligomerization catalyst to provide a second-stage oligomerized stream to provide the oligomerized stream. An embodiment of the disclosure is one, any or all of prior embodiments in this paragraph up through the third embodiment in this paragraph further comprising stripping naphtha and lighter materials from C9+ hydrocarbons in the stripping step.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for oligomerizing an olefin stream comprising:
oligomerizing a charge olefin stream in the presence of a diluent stream over an oligomerization catalyst to produce an oligomerized stream;
hydrogenating said oligomerized stream to provide a distillate stream;
stripping said distillate stream to provide a stripped stream; and
taking said diluent stream from said stripped stream.

2. The process of claim 1 wherein said oligomerizing step further comprises oligomerizing said charge olefin stream in the presence of said diluent stream in a first-stage oligomerization reactor to provide a first-stage oligomerized stream and oligomerizing said first-stage oligomerized stream over a second-stage oligomerization catalyst to provide a second-stage oligomerized stream to provide said oligomerized stream.

3. The process of claim 1 further comprising stripping naphtha and lighter materials from C9+ hydrocarbons in said stripping step.

4. The process of claim 3 further comprising separating an intermediate stream comprising C8+ hydrocarbons from said distillate stream in said stripping step.

5. The process of claim 4 further comprising fractionating said intermediate stream and a jet fractionation feed stream taken from said stripped stream in a jet fractionation column.

6. The process of claim 3 further comprising reboiling a reboil stripper bottoms stream.

7. The process of claim 5 further comprising taking a jet fuel cut from a side or a condensed overhead of said jet fractionator column.

8. The process of claim 5 further comprising operating the jet fractionation column at vacuum.

9. The process of claim 5 further comprising reboiling said jet fractionator by heat exchange with a jet reboil stream taken from said stripped stream.

10. The process of claim 1 further comprising splitting said oligomerized stream into a light olefin stream and a heavy oligomerized stream in an olefin splitter column and taking a recycle olefin stream from said light olefin stream and charging said recycle olefin stream to said oligomerizing step.

11. The process of claim 10 further comprising reboiling said olefin splitter column by heat exchanging a heavy oligomerized stream with a hot oil stream taken from said stripped stream.

12. The process of claim 11 further comprising dealkanizing said oligomerized stream before splitting said oligomerized stream wherein said splitting said oligomerized stream further comprises splitting a dealkanized oligomerized stream.

13. The process of claim 12 further comprising reboiling a dealkanized stream by heat exchange with a cooled hot oil after heat exchange with said heavy oligomerized stream.

14. The process of claim 10 wherein said hydrogenating step comprises hydrogenating said heavy oligomerized stream.

15. A process for oligomerizing an olefin stream comprising:
oligomerizing A charge olefin stream in the presence of a diluent stream in a first-stage oligomerization reactor to provide a first-stage oligomerized stream and oligomerizing said first-stage oligomerized stream over a second-stage oligomerization catalyst to provide a second-stage oligomerized stream to provide an oligomerized stream;
hydrogenating said oligomerized stream to provide a distillate stream;
stripping said distillate stream to provide a stripped stream; and
taking said diluent stream from said stripped stream.

16. The process of claim 15 further comprising stripping naphtha and lighter materials from C9+ hydrocarbons in said stripping step.

17. The process of claim 16 further comprising separating an intermediate stream comprising C8+ hydrocarbons from said distillate stream in said stripping step.

18. A process for oligomerizing an olefin stream comprising:
oligomerizing a charge olefin stream in the presence of a diluent stream over an oligomerization catalyst to produce an oligomerized stream;
hydrogenating said oligomerized stream to provide a distillate stream;
stripping said distillate stream to provide a stripped stream and an intermediate stream comprising C8+ hydrocarbons; and
taking said diluent stream from said stripped stream.

19. The process of claim 18 wherein said oligomerizing step further comprises oligomerizing said charge olefin stream in the presence of said diluent stream in a first-stage oligomerization reactor to provide a first-stage oligomerized stream and oligomerizing said first-stage oligomerized stream over a second-stage oligomerization catalyst to provide a second-stage oligomerized stream to provide said oligomerized stream.

20. The process of claim 1 further comprising stripping naphtha and lighter materials from C9+ hydrocarbons in said stripping step.
